(19) United States
(12) Patent Application Publication
(10) Pub. No.: US 2025/0092312 A1
(43) Pub. Date: Mar. 20, 2025

(54) ANTIPOLYMERANT COMPOSITIONS WITH BENZOQUINONE OR NAPHTHOQUINONE AND OXYGENATED AMINOPHENOL AND METHODS OF USING

(71) Applicant: Ecolab USA Inc., St. Paul, MN (US)

(72) Inventors: Jonathan Masere, Richmond, TX (US); Ashish Dhawan, Aurora, IL (US)

(21) Appl. No.: 18/885,360

(22) Filed: Sep. 13, 2024

Related U.S. Application Data
(60) Provisional application No. 63/538,430, filed on Sep. 14, 2023.

(51) Int. Cl. C09K 15/24; C08F 2/40

(52) U.S. Cl. CPC C09K 15/24; C08F 2/40

(57) ABSTRACT

Described are compositions and methods for inhibiting and retarding polymerization of a monomer composition, which use a naphthoquinone and an oxygenated aminophenol. When present in a monomer-containing composition, the combination of the naphthoquinone and oxygenated aminophenol provides an unexpected beneficial antipolymerant effect inhibiting unwanted formation of fouling polymer. The antipolymerant combination can be added to a hydrocarbon composition, such as a petroleum, that can include a polymerizable components, such as styrene, where the hydrocarbon composition is purified or processed, such as in the case of distillation.

### CROSS REFERENCE TO RELATED APPLICATION

This application claims the benefit of U.S. Provisional Patent Application Ser. No. 63/538,430, filed Sep. 14, 2023, the disclosure of which is incorporated by reference herein in its entirety.

### TECHNICAL FIELD

The invention is directed to compositions and use of a benzoquinone or naphthoquinone in combination with an oxygenated aminophenol for reducing the formation of polymers in a monomer-containing composition.

### BACKGROUND

Ethylenically unsaturated monomers, such as vinyl aromatic monomers like styrene, can be present in processing streams or in refined products made by various chemical industrial processes. However, these monomer types may undesirably polymerize through radical polymerization especially at elevated temperature. As a result, solid deposits of polymer can form on the surface of the process equipment during industrial manufacture, processing, handling, or storage. The resulting polymers can be problematic and lead to equipment “fouling” and product contamination. Accordingly, this can necessitate treating the apparatus to remove the polymer, or may necessitate processing steps that require shutting down operations to physically or mechanically remove the deposited polymer. Equally important, the polymer material contaminates processed streams thereby necessitating steps to remove said contaminants from compositions streams or stored compositions. Consequently, these undesirable polymerization reactions result in a loss in production efficiency because they consume valuable reagents and additional steps may be required to clean equipment and/or to remove the undesired polymers. Undesired polymerization reactions are particularly problematic in compositions having vinyl aromatic monomers.

To minimize undesired polymerization reactions, compounds that act as antipolymerants are often added to process streams or stored compositions. Two categories of antipolymerants have been developed to minimize unwanted polymerization reactions: polymerization inhibitors and polymerization retarders.

Polymerization inhibitors inhibit polymerization reactions from occurring. However, these compounds are generally consumed rapidly. For example, in cases of emergency due to mechanical or processing problems, and where more inhibitor cannot be added, previously added inhibitor will be rapidly consumed. Subsequently, unwanted polymerization reactions will then rapidly recur.

Polymerization retarders, while they slow down the rate of polymerization reactions, are not as effective as polymerization inhibitors. Polymerization retarders, however, are usually not consumed as quickly as polymerization inhibitors so they tend to be more useful in cases of emergency shutdown of operations.

Retarders such as sulfur and dinitrophenol (DNP) compounds such as 2,6-dinitrophenol, 2,4-dinitrocresol, and 2-sec-butyl-4,6-dinitrophenol (DNBP) have found use in antipolymerant methodologies. However, DNP and sulfur retarders release NOx and SOx gases, making their use problematic. Furthermore, DNP-based retarders are highly toxic, which is a concern during handling.

Another class of compounds designed to function as a safer substitute for DNP retarders is based on quinone methide chemistry. Quinone methides slow the rate of polymer formation under static conditions and do not need to be frequently re-fed into the process stream, but some quinone methide compounds do not exhibit good stability. Examples of quinone methide compounds are in U.S. Pat. Nos. 4,003,800, 5,583,247, and 7,045,647.

Another chemistry used for antipolymerant technologies for high-temperature processing of vinylic monomers is 4-hydroxy-2,2,6,6-trimethyl (HTEMPO). Though effective, HTEMPO and its derivatives are efficient inhibitors, but are not effective retarders.

Technical challenges remain in this area of technology relating to efficacy of polymerization inhibitors and retarders, as well as stability and safety concerns. Further, the disclosure is associated with the finding that while desirable to combine polymerization inhibitors and retarders in the same composition, mixtures often suffer from compatibility issues which reduce effectiveness of both the inhibitor and the retarder activities.

Realizing issues associated with the current state of the art of antipolymerants, the current technology of the application relates to a combination composition that is safer and efficacious as an antipolymerant in general and, a retarder in particular.

### SUMMARY

Disclosed herein are compositions and methods for reducing formation of polymers in a composition that includes, or that can form monomers, such as vinylic monomers. Methods of the disclosure use a combination of benzoquinone or naphthoquinone and an oxygenated aminophenol as an antipolymerant formulation, wherein the combination formulation demonstrates an unexpected beneficial antipolymerant effect, such as a synergistic antipolymerant effect, when used together. The mixture of benzoquinone or naphthoquinone in combination with an oxygenated aminophenol can provide excellent activity in inhibiting unwanted polymerization of monomers such as styrene in various applications, such as in synthesis, refinement, or storage. By using the inventive combination benzoquinone or naphthoquinone and oxygenated aminophenol, polymer contamination can be reduced and additional processing steps can be minimized or avoided. In addition, by functioning to inhibit and retard polymerization, the mixture can minimize buildup of unwanted polymer on processing or storage apparatus (“fouling”), and accordingly reduce maintenance costs of such equipment. Moreover, the inventive combination allows the reduction of reagents added to the treated (e.g., hydrocarbon) composition providing higher levels of purity of refined products and reducing overall costs of the product, as well as costs associated with its production. Further, the inventive combination can avoid use of other antipolymerant compounds that are unsafe for use, costly, that have poor effectiveness, or combinations thereof.

Also, in certain embodiments, the benzoquinone or naphthoquinone and oxygenated aminophenol can advantageously be combined in a single package formulation for addition to a composition which can include, or can form monomer, wherein the combination demonstrates an unexpected beneficial antipolymerant effect, such as a synergistic antipolymerant effect, in use.

In embodiments, the disclosure provides a method for inhibiting the polymerization of monomers in a monomer-containing composition. The method includes a step or steps of adding components that include a benzoquinone or naphthoquinone and an oxygenated aminophenol to a composition that is capable of forming a polymerizable monomer. The oxygenated aminophenol compound is of Formula I:

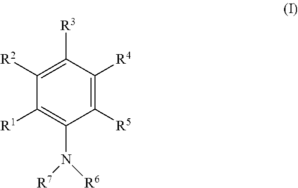

wherein at least one of —R¹, —R², —R³, —R⁴, and —R⁵ is or are —OH, and wherein any one or more of —R¹, —R², —R³, —R⁴, and —R⁵ that is not —OH is or are selected from the group consisting of hydrogen, alkyl, aryl, alkyl aryl and aryl alkyl, or any two adjacent groups of —R¹, —R², —R³, —R⁴, and —R⁵ that are not —OH form one or more ring structures. R⁶ and R⁷ are, independently, carbon-containing groups, and at least one of R⁶ and R⁷ comprises one or more oxygen atom(s) separated from the N atom by one or more carbon atoms. Present together in the composition, the benzoquinone or naphthoquinone and an oxygenated aminophenol inhibit the polymerization of the polymerizable monomer.

In some embodiments, in the oxygenated aminophenol one or both of R⁶ and R⁷ are of the formula: —R⁹OR¹⁰, wherein R⁹ comprises a hydroxylated hydrocarbylene group, optionally substituted, and R¹⁰ comprises a hydrocarbyl group, optionally substituted. Exemplary oxygenated aminophenols include bis[(alkoxyhydroxyalkylene)amino]phenols.

Exemplary benzoquinones include, but are not limited to, 1,4-benzoquinone and 1,2-benzoquinone and alkylated and aminated derivates thereof, and exemplary naphthoquinones include, but are not limited to, 1,4-naphthoquinone, 1,3-naphthoquinone, 1,2-naphthoquinone, and alkylated and aminated derivatives thereof.

In exemplary embodiments, the method can include adding the benzoquinone or naphthoquinone and the oxygenated aminophenol components to a hydrocarbon composition, such as a hydrocarbon composition that is subjected to purification or processing of one or more hydrocarbon components of the composition. The hydrocarbon composition can be derived from a petroleum material and can include a polymerizable components such as styrene, or one or more components capable for forming a polymerizable component. For example, the benzoquinone or naphthoquinone and the oxygenated aminophenol components can be added to a hydrocarbon composition undergoing a distillation step, or can be added to a refined composition prior to its storage or transport.

In other embodiments, the disclosure provides an additive composition that includes the benzoquinone or naphthoquinone, the oxygenated aminophenol, and a suitable solvent or solvent system. The naphthoquinone and the oxygenated aminophenol can be present in predetermined concentrated amounts in the additive composition so that when it is added to a monomer-containing composition, the concentrated amounts are diluted to working amounts that act in an unexpected (e.g., synergistic) manner to inhibit monomer polymerization.

### DETAILED DESCRIPTION

Although the present disclosure provides references to preferred embodiments, persons skilled in the art will recognize that changes may be made in form and detail without departing from the spirit and scope of the invention. Reference to various embodiments does not limit the scope of the claims attached hereto. Additionally, any examples set forth in this specification are not intended to be limiting and merely set forth some of the many possible embodiments for the appended claims.

Additional advantages and novel features of the invention will be set forth in part in the description that follows, and in part will become apparent to those skilled in the art upon examination of the following, or may be learned through routine experimentation upon practice of the invention.

The disclosure provides methods and compositions that use a benzoquinone or naphthoquinone and an oxygenated aminophenol, and in combination are able to be highly effective at preventing the formation of unwanted formation of polymer in a composition that includes monomer. The observed antipolymerant effect is shown to be unexpectedly beneficial, meaning that the degree of inhibition of polymer formation is greater when the benzoquinone/naphthoquinone and oxygenated aminophenol, are used in combination as compared to the degree of polymerization inhibition when the benzoquinone/naphthoquinone and oxygenated aminophenol are used separately, operating under the same total antipolymerant concentrations.

An “unexpected beneficial antipolymerant combination effect” can be observed when the combination of two antipolymerants (a first antipolymerant and a second antipolymerant) provides a level of polymerization inhibition at a total antipolymerant concentration that is greater than the level of polymerization inhibition when either the first or the second antipolymerant is used alone at the amount of total antipolymerant concentration based on the combination. As an example, a first antipolymerant used at a first concentration (e.g., 200 ppm) and which provides an antipolymerant activity with a level of polymerization (e.g., polystyrene formation) at “80”, and a second antipolymerant is also used at a first concentration (e.g., 200 ppm) and which provides an antipolymerant activity with a level of polymerization (e.g., polystyrene formation) at “90”, an “expected” level of antipolymerant activity when the first and second antipolymerants are used in combination at a total concentration of 200 ppm (e.g., 100 ppm each first and second antipolymerants) would be “85” (the average of the levels of polymerization using the first and second antipolymerants). Therefore, if the combination of first and second antipolymerants at the total concentration is less than “85” (the average being expected), or especially less than “80” (the first antipolymerant), then the combination of the antipolymerants is considered to provide an “unexpected” antipolymerant activity, as each antipolymerant is not antagonistic to the other, and the combination performs better than the average of both, or better than either first or second antipolymerant at comparable concentrations. This unexpected level of inhibition of polymerization when first and second antipolymerants are used in combination can optionally be referred to as “synergistic”, and the combination can have “synergistic antipolymerant activity”. A test for unexpected antipolymerant activity can also be performed even if the first and second antipolymerants are used at different concentrations in combination. Here, an “expected” antipolymerant activity again can be calculated based on an average of the first and second antipolymerants used individually at the total antipolymerant concentration. The beneficial antipolymerant activity of the benzoquinone or naphthoquinone and oxygenated aminophenol combination can be caused by the ability of the combination to very effectively inhibit and retard formation of polymer in a monomer composition, wherein such activity can be observed over a period of time, such as exemplified herein.

The ability of the inventive combination of the benzoquinone/naphthoquinone and oxygenated aminophenol can be expressed in terms of a percent (%) reduction as compared to either the benzoquinone/naphthoquinone and oxygenated aminophenol used at the total concentration, or the average of the benzoquinone/naphthoquinone and oxygenated aminophenol used individually at the total concentration. For example, using testing methods, such as described herein, formation of polymer (measured as % w/w) in a monomer composition containing either the benzoquinone/naphthoquinone and oxygenated aminophenol at a total concentration can be determined under conditions promoting polymer formation, and optionally over a period of time. The amount of polymer formation in a combination composition of benzoquinone/naphthoquinone and oxygenated aminophenol (each used at a half of the total concentration) is then determined, and compared to either the average of benzoquinone/naphthoquinone and oxygenated aminophenol, or the benzoquinone/naphthoquinone and oxygenated aminophenol, used individually, at the total concentration. In embodiments, the combination shows a reduction in polymerization of 10% or greater, 20% or greater, 30% or greater, 40% or greater, 50% or greater, 60% or greater, 70% or greater, or 80% or greater, such as up to about 90% or greater, or a reduction in an % amount in the range of any of these numbers, as compared to the average, or as compared to either the of benzoquinone/naphthoquinone and oxygenated aminophenol used individually at the total concentration.

The unexpected (e.g., synergistic) antipolymerant effect can be observed, for example, when the combination of the benzoquinone/naphthoquinone and oxygenated aminophenol are introduced into a hydrocarbon composition that includes a polymerizable monomer, or a compound capable for forming a polymerizable monomer, such as a hydrocarbon composition that includes styrene. The combination can be introduced into a hydrocarbon composition that is undergoing refining, or can be introduced into a refined hydrocarbon composition, such as one that is being transported or stored.

“Antipolymerants” as used herein are compounds that can reduce the formation of polymers from one or more radically polymerizable compounds. Antipolymerants may be more specifically categorized as polymerization inhibitors and polymerization retarders. A polymerization inhibitor, in the presence of polymerizable monomers, inhibits the formation of a polymer from those monomers during the induction time. After the induction time has lapsed, the polymer's formation occurs at substantially the same rate that it would form at in the absence of the polymerization inhibitor. A polymerization retarder does not exhibit an induction time, but instead once added to a polymerizable monomer composition reduces the rate at which the formation of the polymer occurs relative to the rate at which it would have formed in the absence of the composition of matter. Polymerization inhibitors, as opposed to polymerization retarders, are generally consumed rapidly. Polymerization retarders, while they slow down the rate of polymerization reactions, are not as effective as polymerization inhibitors. Polymerization retarders, however, are usually not consumed as quickly as polymerization inhibitors. The inventive combination of the benzoquinone/naphthoquinone and oxygenated aminophenol components can provide a beneficial antipolymerant effect, thereby reducing the formation of polymers in the presence of a monomer-containing composition, by inhibiting and retarding polymer formation. This beneficial activity can be observed over a period of time, such as exemplified herein.

Aspects of the disclosure provide a composition for inhibiting monomer polymerization that include a benzoquinone/naphthoquinone and oxygenated aminophenol. A composition that includes these components (and any one or more optional component) can be in a desired form, such as in a liquid form, a dry form, or as a suspension or dispersion. The benzoquinone/naphthoquinone and oxygenated aminophenol can be in desired physical states in the composition, such as in a dissolved state, in a partially dissolved state, in a suspended state, or in a dry mixture. Also, the benzoquinone/naphthoquinone and oxygenated aminophenol can be in desired forms in the composition, such as optionally in particulate forms. If one or more of the components is in a particulate form, the particles can optionally be described in terms of particle size (e.g., particles of a size range) and/or shape. The form of the composition and the state of the components therein can be chosen by selection of benzoquinone/naphthoquinone and oxygenated aminophenol, with an understanding of the physical property of each compound. The form of the composition and the state of the components therein can also be affected by the inclusion of one or more optional components, such as a solvent, or solvent mixture, or other excipient compounds like surfactants, dispersants, etc. The form of the composition and the state of the components therein can also be affected by temperature, and composition properties may optionally be described in circumstances at a particular temperature (e.g., at a storage temperature such as 5° C. or below, at room temperature (25° C.), or at a temperature used for monomer synthesis and/or processing (e.g., about 100° C. or greater, about 150° C., about 175° C., etc.).

A composition including the benzoquinone/naphthoquinone and oxygenated aminophenol can optionally include other components in the composition (e.g., described in terms of a composition “comprising” the benzoquinone/naphthoquinone and oxygenated aminophenol). For example, such compositions can include other components such as a solvent, surfactants, dispersants, etc. If an optional component is present in the composition, it may be described in terms of a weight amount relative to one or more of the benzoquinone/naphthoquinone and oxygenated aminophenol compounds in the composition. The optional component may be present in a weight amount greater than, or an amount less than, any one of the benzoquinone/naphthoquinone and oxygenated aminophenol, or the total amount of benzoquinone/naphthoquinone and oxygenated aminophenol.

As used herein, the term “optional” or “optionally” means that the subsequently described object (e.g., compound), event (e.g., processing step), amount, or circumstance may, but need not occur, and that the description includes instances where the object, event, amount, or circumstance occurs and instances in which it does not.

Compositions of the disclosure can include those recited compounds and optionally can include other components in the composition but in very small amounts (e.g., described in terms of a composition “consisting essentially of” the recited components). For example, such compositions can include one or more other components but not in an amount that is greater than about 1% (wt), about 0.5% (wt), or about 0.1% (wt), of the total composition. A composition that consists essentially of the benzoquinone/naphthoquinone, oxygenated aminophenol, and a solvent can optionally include one or more other components, but in an amount less than about 1% (wt) of the total composition. In a composition “consisting of” the recited components there is no other measurable amount of component other than the recited component.

Likewise, the chemistries of compounds of the disclosure, including the benzoquinone/naphthoquinone and oxygenated aminophenol can, in some embodiments, be described in terms of the compound “consisting of” certain atoms or certain chemical groups. For example, in embodiments of the disclosure a compound, such as the oxygenated aminophenol component, can consist of carbon (C), hydrogen (H), oxygen (O), and nitrogen (N), and will not have any other types of atoms aside from C, H, O, and N, in the compound. As another example, a compound consisting of a hydrocarbyl group, a hydroxyl group, and an amine group will not have any other chemical groups aside from these.

As used herein, the terms “substantially” and “consisting essentially of” modifying, for example, the type or quantity of an ingredient in a composition, a property, a measurable quantity, a method, a position, a value, or a range, employed in describing the embodiments of the disclosure, refers to a variation that does not affect the overall recited composition, property, quantity, method, position, value, or range thereof in a manner that negates an intended composition, property, quantity, method, position, value, or range. Examples of intended properties include, solely by way of nonlimiting examples thereof, dispersibility, stability, rate, solubility, and the like; intended values include weight of a component added, concentration of components added, and the like. The effect on methods that are modified include the effects caused by variations in type or amount of materials used in a process, variability in machine settings, the effects of ambient conditions on a process, and the like wherein the manner or degree of the effect does not negate one or more intended properties or results; and like proximate considerations. Where modified by the term “substantially” or “consisting essentially of,” the claims appended hereto include equivalents to these types and amounts of materials.

As used herein, the term “about” modifying, for example, the quantity of an ingredient in a composition, concentration, volume, process temperature, process time, yield, flow rate, pressure, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods, and like proximate considerations. The term “about” also encompasses amounts that differ due to aging of a formulation with a particular initial concentration or mixture, and amounts that differ due to mixing or processing a formulation with a particular initial concentration or mixture. Where modified by the term “about” the claims appended hereto include equivalents to these quantities. Further, where “about” is employed to describe any range of values, for example “about 1 to 5” the recitation means “1 to 5” and “about 1 to about 5” and “1 to about 5” and “about 1 to 5” unless specifically limited by context.

Some R groups in formulas of the disclosure can include hydrocarbon-containing groups such as alkyl groups, including linear, branched, and cyclic alkyl groups, aryl groups, alkyl aryl groups (e.g., ethyl-benzyl), aryl alkyl groups (e.g., phenyl-propyl), and combinations thereof. Cyclic alkyl or aryl groups can have fused structures such as decahydronaphthalene, naphthalene, tetradecahydro-anthracene, anthracene, etc. In some embodiments, hydrocarbon groups of formulas of the disclosure can be defined by the number of carbon atoms in the group (e.g., R group), such as 1-18 carbons, 2-18 carbons, 3-17 carbons, 4-16 carbons, 5-14 carbons, or 6-12 carbon atoms, or 1-10 carbons, 1-8 carbons, 1-6 carbons, 1-5 carbons, 1-4 carbons, or 1-3 carbons.

Aminophenol compounds of the current disclosure can be described with reference Formula I:

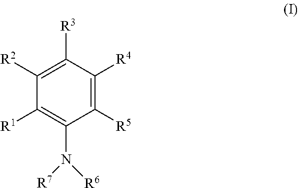

wherein at least one of —R¹, —R², —R³, —R⁴, and —R⁵ is or are —OH, and wherein any one or more of —R¹, —R², —R³, —R⁴, and —R⁵ that is not —OH is or are selected from the group consisting of hydrogen, alkyl, aryl, alkyl aryl and aryl alkyl, or any two adjacent groups of —R¹, —R², —R³, —R⁴, and —R⁵ that are not —OH form one or more ring structures; wherein R⁶ and R⁷ are, independently, carbon-containing groups, and at least one of R⁶ and R⁷ comprises one or more oxygen atom(s) separated from the N atom by one or more carbon atoms;

In embodiments, one or more of —R¹, —R², —R³, —R⁴, and —R⁵ is or are —OH, and —R¹, —R², —R³, —R⁴, and —R⁵ that are not —OH are —H. As such, Formula I can be based on compounds such as aminophenol, aminopyrocatechol, aminoresorcinol, aminohydroquinone, aminohydroxyhydroquinone, and aminophlorolucitol.

Alternatively, two adjacent groups of —R¹, R², —R³, —R⁴, and —R⁵ that are not —OH form one or more ring structures, for example the compound can include a bicyclic or a tricyclic structure, including one, two, or three 6-membered rings, or a 6-membered ring fused to a 5-membered ring. For example, the nitrogen- and oxygen-containing aromatic can be based on hydroxyl-containing fused aromatic chemistries such as aminonaphthol, aminohydroxyanthracene, or aminoindenol.

In embodiments, the aminophenol compounds of the disclosure include an ether chemistry wherein one or both of R⁶ and R⁷ are of the formula: —R⁹OR¹⁰. In —R⁹OR¹⁰, R⁹ is hydrocarbylene group, optionally substituted (such as with an alkyl group, alkoxy group, etc.,), and R¹⁰ is hydrocarbyl group, optionally substituted. One or both of R⁹ and R¹⁰ can have an amount of carbon atoms in the range of 1-18, 1-12, 1-8, 1-6, or 1-3.

In embodiments, R⁹ is of the sub-formula —(CR²⁴R²⁵)ₛ—, wherein R²⁴R²⁵ are independently selected from H, R¹⁰, and OR¹⁰, wherein s is an integer in the range of 1-12, 1-6, or preferably 1-3.

For example, R⁹ can be a linear or branched C1-C6 hydrocarbylene group, such as one selected from the group consisting of methylene, ethylene, propylene, isopropylene, butylene, isobutylene, sec-butylene, tert-butylene, pentylene, and hexylene.

For example, R¹⁰ can be selected from linear, branched, or cyclic alkyl, aryl, alkyl-aryl, and aryl-alkyl C1-C12 groups. Exemplary R¹⁰ groups include those such as methyl, ethyl, propyl, isopropyl, butyl, isobutyl, sec-butyl, tert-butyl, pentyl, cyclopentyl, isopentyl, neopentyl, hexyl, cyclohexyl, 1-, 2-, and 3-methylbutyl, 1,1-, 1,2-, or 2,2-dimethylpropyl, 1-ethyl-propyl, 1-, 2-, 3-, or 4-methylpentyl, 1,1-, 1,2-, 1,3-, 2,2-, 2,3-, or 3,3-dimethylbutyl, 1- or 2-ethylbutyl, 1-ethyl-1-methylpropyl, and 1,1,2- or 1,2,2-trimethylpropyl, methylcyclopentyl; heptyl, 2-methylhexyl, 3-methylhexyl, 4-methylhexyl, 5-methylhexyl, 3-ethylpentyl, 2,2,3-trimethylbutyl, 2,2-dimethylpentyl, 2,3-dimethylpentyl, 2,4-dimethylpentyl, 3,3-dimethylpentyl, 3,4-dimethylpentyl, 4,4-dimethylpentyl, cycloheptyl, 1-methylcyclohexyl, and 2-methylcyclohexyl; octyl, 2-methylheptyl 3-methylheptyl, 4-methylheptyl, 2-ethylhexyl, 3-ethylhexyl, 4-ethylhexyl, 5-ethylhexyl, 2,2-dimethylhexyl, 2,3-dimethylhexyl, 2,4-dimethylhexyl, 2,5-dimethylhexyl, 3,3-dimethylhexyl, 3,4-dimethylhexyl, 3-ethyl-2-methylpentyl, 3-ethyl-3-methylpentyl, 2,2,3-trimethylpentyl, 2,2,4-trimethylpentyl, 2,3,3-trimethylpentyl, 2,3,4-trimethylpentyl, and 2,2,3,3-tetramethylbutyl.

In exemplary embodiments, the aminophenol is a compound selected from the group consisting of:
4-bis[(methoxymethyl)amino]phenol, 4-bis[(2-methoxyethyl)amino]phenol, 4-bis[(3-methoxypropyl)amino]phenol, 4-bis[(4-methoxybutyl)amino]phenol, 4-bis[(5-methoxypentyl)amino]phenol, 4-bis[(6-methoxyhexyl)amino]phenol, 4-bis[(methoxyphenyl)amino]phenol;
4-bis[(ethoxymethyl)amino]phenol, 4-bis[(2-ethoxyethyl)amino]phenol, 4-bis[(3-ethoxypropyl)amino]phenol, 4-bis[(4-ethoxybutyl)amino]phenol, 4-bis[(5-ethoxypentyl)amino]phenol, 4-bis[(6-ethoxyhexyl)amino]phenol, 4-bis[(ethoxyphenyl)amino]phenol;
4-bis[(propoxymethyl)amino]phenol, 4-bis[(2-propoxyethyl)amino]phenol, 4-bis[(3-propoxypropyl)amino]phenol, 4-bis[(4-propoxybutyl)amino]phenol, 4-bis[(5-propoxypentyl)amino]phenol, 4-bis[(6-propoxyhexyl)amino]phenol, 4-bis[(propoxyphenyl)amino]phenol;
4-bis[(butoxymethyl)amino]phenol, 4-bis[(2-butoxyethyl)amino]phenol, 4-bis[(3-butoxypropyl)amino]phenol, 4-bis[(4-butoxybutyl)amino]phenol, 4-bis[(5-butoxypentyl)amino]phenol, 4-bis[(6-butoxyhexyl)amino]phenol, and 4-bis[(butoxyphenyl)amino]phenol.

In some embodiments, in the aminophenol of Formula I, one or both of R⁶ and R⁷ include both ether and hydroxyl groups. For example, one or both of R⁶ and R⁷ are of the formula: —R⁹OR¹⁰, where R⁹ is a hydroxylated hydrocarbylene group, and R¹⁰ is hydrocarbyl group, optionally substituted. For example, R⁹ can be C1-C8 hydrocarbylene with a single hydroxyl group, such as one selected from the group consisting of:

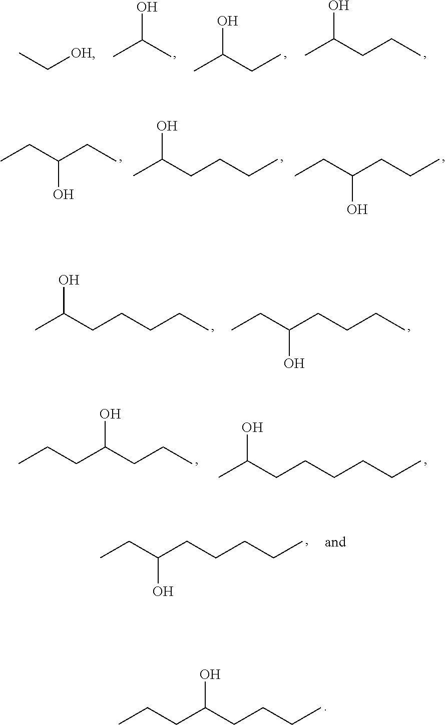

In exemplary embodiments, the aminophenol compound having ether and hydroxylated hydrocarbylene groups can be a compound selected from the group consisting of:
4-bis[(2-methoxy-1-hydroxy-ethyl)amino]phenol, 4-bis[(3-methoxy-2-hydroxy-propyl)amino]phenol, 4-bis[(4-methoxy-2-hydroxy-butyl)amino]phenol, 4-bis[(5-methoxy-2-hydroxy-pentyl)amino]phenol, 4-bis[(6-methoxy-2-hydroxy-hexyl)amino]phenol, 4-bis[(methoxyhydroxyphenyl)amino]phenol;
4-bis[(2-ethoxy-1-hydroxy-ethyl)amino]phenol, 4-bis[(3-ethoxy-2-hydroxy-propyl)amino]phenol, 4-bis[(4-ethoxy-2-hydroxy-butyl)amino]phenol, 4-bis[(5-ethoxy-2-hydroxy-pentyl)amino]phenol, 4-bis[(6-ethoxy-2-hydroxy-hexyl)amino]phenol, 4-bis[(ethoxyhydroxyphenyl)amino]phenol;
4-bis[(2-propoxy-1-hydroxy-ethyl)amino]phenol, 4-bis[(3-propoxy-2-hydroxy-propyl)amino]phenol, 4-bis[(4-propoxy-2-hydroxy-butyl)amino]phenol, 4-bis[(5-propoxy-2-hydroxy-pentyl)amino]phenol, 4-bis[(6-propoxy-2-hydroxy-hexyl)amino]phenol, 4-bis[(propoxyhydroxyphenyl)amino]phenol,
4-bis[(2-butoxy-1-hydroxy-ethyl)amino]phenol, 4-bis[(3-butoxy-2-hydroxy-propyl)amino]phenol, 4-bis[(4-butoxy-2-hydroxy-butyl)amino]phenol, 4-bis[(5-butoxy-2-hydroxy-pentyl)amino]phenol, 4-bis[(6-butoxy-2-hydroxy-hexyl)amino]phenol, and 4-bis[(butoxyhydroxyphenyl)amino]phenol

In some embodiments, in the aminophenol compounds of Formula I, one or both of R⁶ and R⁷ is or are a hydroxylated hydrocarbyl group(s). Exemplary hydroxylated hydrocarbyl group(s) can have an amount of carbon atoms in the range of 1-18, 1-12, 1-8, 1-6, or 1-3, and can be selected from hydroxylated linear, branched, and cyclic alkanol, hydroxylated aryl, hydroxylated alkyl-aryl, and hydroxylated aryl-alkyl.

In embodiments, R⁹ is of the sub-formula —(CR²⁴R²⁵)_{q}(CHOH)(CH₂)_{z}R¹¹, wherein R²⁴ and R²⁵ are independently selected from H, R¹⁰, and OR¹⁰, wherein q and z are independently (−): a covalent bond, or an integer in the range of 1-12, preferably (−) or an integer in the range of 1-6 or 1-3, and wherein R¹¹ is selected from the group consisting of R¹⁰, as described herein, and

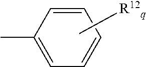

wherein R¹² is independently selected from the group consisting of H and R¹⁰, as described herein, and wherein q is an integer in the range of 1-5. In some embodiments, R⁹ is of the sub-formula —(CH₂)_{q}(CHOH)(CH₂)_{z}R¹¹, wherein the variables have the meanings given herein.

In exemplary embodiments, the aminophenol with a hydroxylated hydrocarbylene group can be a compound selected from the group consisting of:
4-bis[(hydroxymethyl)amino]phenol
4-bis[(1-hydroxyethyl)amino]phenol, 4-bis[(2-hydroxyethyl)amino]phenol
4-bis[(1-hydroxypropyl)amino]phenol, 4-bis[(2-hydroxypropyl)amino]phenol, 4-bis[(3-hydroxypropyl)amino]phenol,
4-bis[(1-hydroxybutyl)amino]phenol, 4-bis[(2-hydroxybutyl)amino]phenol, 4-bis[(3-hydroxybutyl)amino]phenol, 4-bis[(4-hydroxybutyl)amino]phenol,
4-bis[(1-hydroxypentyl)amino]phenol, 4-bis[(2-hydroxypentyl)amino]phenol, 4-bis[(3-hydroxypentyl)amino]phenol, 4-bis[(4-hydroxypentyl)amino]phenol, 4-bis[(5-hydroxypentyl)amino]phenol
4-bis[(1-hydroxyhexyl)amino]phenol, 4-bis[(2-hydroxyhexyl)amino]phenol, 4-bis[(3-hydroxyhexyl)amino]phenol, 4-bis[(4-hydroxyhexyl)amino]phenol, 4-bis[(5-hydroxyhexyl)amino]phenol, 4-bis[(6-hydroxyhexyl)amino]phenol,
4-bis[(1-hydroxyhexyl)amino]phenol, 4-bis[(2-hydroxyhexyl)amino]phenol, 4-bis[(3-hydroxyhexyl)amino]phenol, 4-bis[(4-hydroxyhexyl)amino]phenol, 4-bis[(5-hydroxyhexyl)amino]phenol, 4-bis[(6-hydroxyhexyl)amino]phenol,
4-bis[(2-hydroxy-2-phenylethyl)amino]phenol, 4-bis[(2-hydroxy-3-phenylpropyl)amino]phenol, 4-bis[(3-hydroxy-3-phenylpropyl)amino]phenol, 4-bis[(2-hydroxy-4-phenylbutyl)amino]phenol, 4-bis[(3-hydroxy-4-phenylbutyl)amino]phenol, and 4-bis[(4-hydroxy-4-phenylbutyl)amino]phenol.

Aminophenol compounds of the disclosure can be prepared using methods as described herein and as known in the art. In some modes of practice, and as a general matter, an aryl-group containing reactant having primary amine and hydroxyl groups, such as 4-aminophenol, is reacted with a molar excess (e.g., a two-molar excess) of a carbon and oxygen-containing reactant, capable of reaction with the primary amine groups to provide a product as described herein. The compounds can be reacted in an organic solvent such as an alcohol like methanol, butyl carbitol, and butyl glycol, with reflux at an elevated temperature (e.g., >100° C.).

In some modes of practice, the reactant includes an oxirane group as the amine-reactive group. Oxirane-containing reactants can include desired carbon chemistry and can also include additional oxygen atom(s), such as in the form of ether groups. Exemplary oxirane-containing reactants are glycidyl ethers, such as alkyl glycidyl ethers and alkyl glycidyl ethers.

In some modes of practice, the oxirane-containing reactant is of formula II:

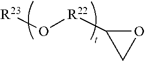

wherein R²² is —(CH₂)_{w}—, wherein w is an integer in the range of 1-3, t is an integer in the range of 1-100, 1-50, 1-25, 1-15, 1-10, 1-5, or t is 2, 3, or 4, and wherein R²³ is R¹⁰, as described herein, optionally substituted with one or more hydroxyl groups.

Methods of preparing the aminophenol compounds of the disclosure, can be found in commonly assigned PCT Application No. WO2020/223224 (Masere and Dhawan).

In some embodiments, compositions and methods of the disclosure include or use a benzoquinone. In embodiments, compositions and method of the disclosure use one or more benzoquinone(s) having chemical structures based on 1,2-benzoquinone and 1,4-benzoquinone architectures according to Formulas III and IV, respectively.

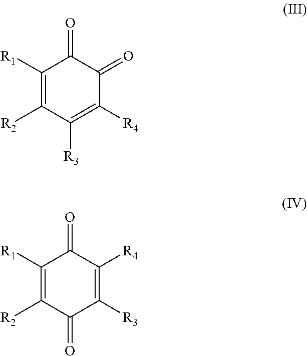

In embodiments, in Formulas III and IV, R¹-R⁴ are —H providing the compounds 1,2-benzooquinone and 1,4-benzoquinone.

In other embodiments, one or more of R¹-R⁴ is/are a chemical group other than-H, such a chemical group selected from the group consisting of alkyl, aryl, alkyl aryl and aryl alkyl groups of 1 to 24 carbon atoms. Exemplary alkyl groups include linear, branched, and cyclic alkyl groups.

Exemplary linear alkyl groups include methyl, ethyl, propyl, butyl, pentyl, hexyl, heptyl, octyl, nonyl, decyl, undecyl, and dodecyl. Exemplary branched alkyl groups include isobutyl, sec-butyl, tert-butyl, 1-, 2-, and 3-methylbutyl, 1,1-, 1,2-, or 2,2-dimethylpropyl, 1-ethyl-propyl, 1-, 2-, 3-, or 4-methylpentyl, 1,1-, 1,2-, 1,3-, 2,2-, 2,3-, or 3,3-dimethylbutyl, 1- or 2-ethylbutyl, 1-ethyl-1-methylpropyl, and 1,1,2- or 1,2,2-trimethylpropyl.

Exemplary benzoquinone species wherein one or more of R¹-R⁴ is/are a chemical group other than-H, include, but are not limited to, 2-methyl-1,4-benzoquinone, 2-ethyl-1,4-benzoquinone, 2-ethyl-5-methyl-1,4-benzoquinone, 2-tert-butyl-1,4-benzoquinone, 3-tert-butyl-1,4-benzoquinone, 2,6-di-tert-butyl-1,4-benzoquinone, 3,5-di-tert-butyl-1,4-benzoquinone, and 2-tert-butyl-5-methyl-1,4-benzoquinone.

In embodiments, the benzoquinone is an aminated benzoquinone. For example, in Formulas III and IV, the benzoquinone can include one or more amine groups, wherein one or both of —R⁵ and/or —R⁶ is or are —NR⁵R⁶, wherein R⁵ and R⁶ are selected from the group consisting of hydrogen, alkyl, aryl, alkyl aryl and aryl alkyl groups of 1 to 24 carbon atoms. Reflecting the positions of carbonyl groups and amine group(s) on the ring, exemplary aminated benzoquinones can have the general chemistries of 2-2-amino, 1,4-benzoquinone, 2,3-diamino, 1,4-benzoquinone, 3,5-diamino, 1,4-benzoquinone, 3-amino, 1,2-benzoquinone, 4-amino, 1,2-benzoquinone, 3,4-diamino, 1,2-benzoquinone, 4,5-diamino, 1,2-benzoquinone, 3,6-diamino, 1,2-benzoquinone, 4,6-diamino, 1,2-benzoquinone.

In embodiments, compositions and methods of the disclosure include or use a naphthoquinone. In embodiments, compositions and method of the disclosure use one or more naphthoquinone(s) having chemical based on 1,4-naphthoquinone and 1,2-naphthoquinone architectures according to Formulas V and VI, respectively.

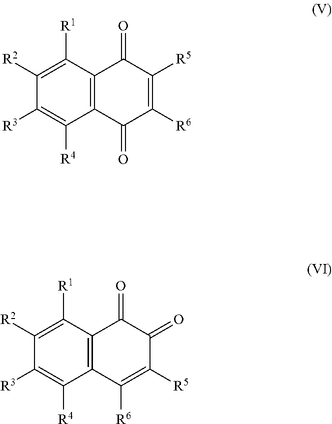

In embodiments, in Formulas V and VI, R¹-R⁶ are —H providing the compounds 1,4-naphthoquinone and 1,2-naphthoquinone.

Alternatively, two adjacent groups of R¹, R², R³, or R⁴ (i.e., R¹ and R²; R² and R³; or R³ and R⁴) form one or more ring structures. This can provide, for example, anthracene, fused ring structures derived from naphthoquinone.

In other embodiments, one or more of R¹-R⁶ are a chemical group other than-H, such a chemical group selected from the group consisting of alkyl, aryl, alkyl aryl and aryl alkyl groups of 1 to 24 carbon atoms. Exemplary alkyl groups include linear, branched, and cyclic alkyl groups. In some cases, two or more of R¹-R⁶ include the same alkyl group, and in other cases one of R¹-R⁶ includes one type of alkyl group, and another R¹-R⁶ group include a different type of alkyl group. Exemplary naphthoquinone species wherein one or more of R¹-R⁶ is/are a chemical group other than-H, include, but are not limited to, 2-tert-butyl-1,4-naphthoquinone, 2,7-di-tert-butyl-1,4-naphthoquinone, 6,7-di-t-butyl-1,4-naphthoquinone, 2-isopropyl-3-tert-butyl-1,4-naphthoquinone, 2-tert-butyl-6-(cyclohexyloxy)-1,4-naphthoquinone, and 2-tert-butyl-3-methylnaphthoquinone.

In embodiments, the naphthoquinone is an aminated naphthoquinone. For example, in Formula III or IV, the naphthoquinone can include one or more amine groups, wherein one or both of —R⁵ and/or —R⁶ is or are —NR⁵R⁶, wherein R⁵ and R⁶ are selected from the group consisting of hydrogen, alkyl, aryl, alkyl aryl and aryl alkyl groups of 1 to 24 carbon atoms. Exemplary aminated naphthoquinone antipolymerants can have the general chemistries of 2-amino, 1,4-naphthoquinone, 2,3-diamino, 1,4-naphthoquinone, 2-amino, 1,3-naphthoquinone, 4-amino, 1,3-naphthoquinone, 2,4-diamino, 1,3-naphthoquinone, 3-amino, 1,2-naphthoquinone, 4-amino, 1,2-naphthoquinone, and 3,4-diamino, 1,3-naphthoquinone.

Exemplary aminated naphthoquinone are described in Applicant's U.S. Pat. No. 11,312,792 (Masere, Apr. 26, 2022), the disclosure of which is incorporated herein by reference.

Compositions and methods of the disclosure include or use an aminophenol along with a benzoquinone/naphthoquinone, which provide an unexpected (e.g., synergistic) antipolymerant effect with present together in a monomer-containing composition.

Amounts of the aminophenol and the benzoquinone/naphthoquinone, in a composition can be described in various ways, such as by a weight percentage (% wt.) of each component in the composition, or by molar amounts of the compounds. These compounds can also be described in terms of weight ratios, or in terms of relative amounts to one another, in a composition.

In some embodiments, in a composition the aminophenol and the benzoquinone/naphthoquinone are used at a desired molar ratio, such as a ratio in the range of 10:1 to 1:10, in the range of 5:1 to 1:5, in the range of 2.5:1 to 1:2.5, in the range of 2:1 to 1:2, in the range of 1.5:1 to 1:1.5, in the range of 1.25:1 to 1:1.25, or in the range of 1.1:1 to 1:1.1. In some embodiments the aminophenol and the benzoquinone/naphthoquinone, are used at approximately the same molar amounts.

The aminophenol and the benzoquinone/naphthoquinone can be present in a composition with a solvent, or a combination of solvents. A solvent or solvent combination can be chosen so that at least one of the aminophenol and the benzoquinone/naphthoquinone is soluble in the solvent or solvent combination. If the benzoquinone/naphthoquinone is a liquid at ambient conditions, the solvent can be chosen so that it is miscible with the aminophenol.

The composition can also include one or more solvents. Useful solvents include any solvent in which a combination of aminophenol and the benzoquinone/naphthoquinone are soluble or can be stably suspended. In some embodiments, a solvent or solvent combination can be selected from water-soluble or water-miscible solvents such glycol-based solvents and hydrophobic solvents such as aromatic solvents, paraffinic solvents, or mixtures of both.

Exemplary glycol solvents include, but are not limited, C₁-C₈ glycols such as ethylene glycol, propylene glycol, diethylene glycol, and triethylene glycol, ethers of such glycols such as diethylene glycol monobutyl ether, diethylene glycol monomethyl ether, diethylene glycol monoethyl ether, diethylene glycol monobutyl ether, triethylene glycol, triethylene glycol monomethyl ether, liquid polyethylene glycol, dipropylene glycol, dipropylene glycol monomethyl ether, dipropylene glycol monoethyl ether, and a low molecular weight polypropylene glycol and the like and combinations thereof. Commercial solvents such as Butyl Carbitol and Butyl CELLOSOLVE™, which contains primarily Butyl CARBITOL™ which consists primarily of ethylene glycol monobutyl ether may be used and are available from DOW.

Other exemplary hydrophobic solvents include heavy aromatic naphtha, toluene, ethylbenzene, and isomeric hexanes, and mixtures of two or more thereof.

The amount of one or more solvents in a composition that includes the aminophenol and the benzoquinone/naphthoquinone is not particularly limited. In some embodiments, the amount of one or more solvents in the composition can be about 10 wt % to 50 wt %, for example, about 20 wt % to 50 wt %, or about 25 wt % to 50 wt %, or about 10 wt % to 40 wt %, or about 10 wt % to 30 wt %, or about 20 wt % to 40 wt %, or about 25 wt % to 40 wt % of the total composition.

In some embodiments the composition includes the benzoquinone/naphthoquinone in an amount in the range of 5 to 45% (wt); the aminophenol in an amount in the range of 5 to 45% (wt); and a solvent, or solvent combination, in an amount in the range of 10 to 90% (wt). In some embodiments the composition includes the benzoquinone/naphthoquinone in an amount in the range of 15 to 35% (wt); the aminophenol in an amount in the range of 15 to 35% (wt); and a solvent, or solvent combination, in an amount in the range of 30 to 70% (wt). In some embodiments the composition includes the benzoquinone/naphthoquinone in an amount in the range of 20 to 30% (wt); the aminophenol in an amount in the range of 20 to 30% (wt); and a solvent, or solvent combination, in an amount in the range of 30 to 70% (wt). Compositions including benzoquinone/naphthoquinone and oxygenated aminophenol at concentrations that are higher than “working” concentrations of naphthoquinone or the oxygenated aminophenol (typically measured in ppm, can be referred to as “concentrates” or “stock” compositions.

Compositions of the disclosure can be made using any desired method. In some modes of making, a solution of the aminophenol or benzoquinone/naphthoquinone with solvent can first be obtained by a user, such as a commercial preparation, and then either the aminophenol or benzoquinone/naphthoquinone is added subsequently, such as in a point of use procedure.

A composition that includes a mixture of the aminophenol and the benzoquinone/naphthoquinone, optionally with solvent, can be provided as a “shelf-stable” composition, and then subsequently used in a process to inhibit polymerization of monomers. For example, methods of the disclosure can include a step of preparing a composition of the aminophenol and the benzoquinone/naphthoquinone, and then storing the composition for a period of time, and then using the composition in a process to inhibit polymerization of monomers.

Methods of abating the polymerization of monomers in a monomer-containing composition can be carried out by adding the aminophenol and the benzoquinone/naphthoquinone to a composition that includes a polymerizable monomer. The aminophenol and the benzoquinone/naphthoquinone inhibit the polymerization of the polymerizable monomer at a desired total concentration, and their beneficial use in combination provide a degree of polymerization inhibition that is greater than either of the compounds used individually at the total concentration. In other words, the combination of the aminophenol and the benzoquinone/naphthoquinone provide an unexpected (e.g., synergistic) effect to inhibit monomer polymerization, which in turn provides benefits for various processes where inhibiting monomer polymerization is desired.

As noted herein, the aminophenol and the benzoquinone/naphthoquinone can provide an unexpected (e.g., synergistic) effect when used together in a monomer-containing composition, which can in turn eliminate the need for one or more other types of polymerization inhibitors to be used concomitantly with the aminophenol and the benzoquinone/naphthoquinone. Other types of polymerization inhibitors that may optionally be excluded from use at the same time as the aminophenol and the benzoquinone/naphthoquinone, or may be used at very small concentrations, can include nitroxide-, amine oxide-, nitro-, nitroso-, and nitrone-containing compounds.

For example, if even present, any nitroxide-, amine oxide-, nitro-, nitroso-, and nitrone-containing compound can optionally be present in an amount of less than 50 ppm, less than 25 ppm, less than 10 ppm, less than 5 ppm, less than 2.5 ppm, less than 2 ppm, less than 1.5 ppm, less than 1 ppm, less than 0.75 ppm, or less than 0.5 ppm, or at essentially no detectable level, in a working composition with monomer.

Exemplary nitroxide-containing inhibitor include di-tert-butyl nitroxyl, 2,2,6,6-tetramethylpiperidinyl-1-oxyl (TEMPO), 4-hydroxy-2,2,6,6-tetramethylpiperidinyl-1-oxyl (HTMPO), 4-oxo-2,2,6,6-tetramethylpiperidinyl-1-oxyl (OTEMPO), which may be excluded from additive compositions of the disclosure, or not be added to a monomer composition concomitantly with the naphthoquinone and oxygenated aminophenol. These types of compounds may be excluded from use at the same time as the naphthoquinone and oxygenated aminophenol, or may be used at very small concentrations, as noted herein.

Exemplary hydroxylamine-containing polymerization inhibitors include, but are not limited to: 1-hydroxy-2,2,6,6-tetramethylpiperidine (TEMPOH), 1,4-dihydroxy-2,2,6,6-tetramethylpiperidine (HTMPOH), and 1-hydroxy-4-oxo-2,2,6,6-tetramethylpiperidine (OTEMPOH), N,N-diethylhydroxylamine, and N-isopropylhydroxylamine. These types of compounds may be excluded from use at the same time as the oxygenated aminophenol and the benzoquinone/naphthoquinone, or may be used at very small concentrations, as noted herein.

Exemplary nitro-containing polymerization inhibitors include, but are not limited to: nitrobenzene, nitrophenol, dinitrophenol, 2,4-dinitro-6-s-butylphenol, 2,4-dinitro-o-cresol, and diphenyl picrylhydrazyl. These types of compounds may be excluded from use at the same time as the oxygenated aminophenol and the benzoquinone/naphthoquinone, or may be used at very small concentrations, as noted herein.

Exemplary nitroso-containing polymerization inhibitors include, but are not limited to: nitrosobenzene, nitrosophenol, dinitrosophenol, dinitrosotoluene, nitrosophenyl-hydroxylamine. These types of compounds may be excluded from use at the same time as the oxygenated aminophenol and the benzoquinone/naphthoquinone, or may be used at very small concentrations, as noted herein.

Although it is preferred that oxygenated aminophenol and the benzoquinone/naphthoquinone is used to provide an antipolymerant effect without the use of a nitroxide-, amine oxide-, nitro-, nitroso-, and nitrone-containing compound, or optionally with such a compound in a very small amount, the methods of the disclosure do not preclude using a monomer-containing composition that has been previously treated with a nitroxide-, amine oxide-, nitro-, nitroso-, or nitrone-containing compound, nor does it preclude a method wherein such a compound is added at a time after the oxygenated aminophenol and the benzoquinone/naphthoquinone treatment. For example, the disclosure contemplates hydrocarbon and monomer-containing compositions previously treated with an, e.g., nitroxide compound, and then after a period of time wherein the nitroxide compound loses its effectiveness, the oxygenated aminophenol and the benzoquinone/naphthoquinone are then added to the hydrocarbon and monomer-containing composition. Optionally, the disclosure also contemplates hydrocarbon and monomer-containing compositions that are treated with the oxygenated aminophenol and the benzoquinone/naphthoquinone, and then subsequently treated with a compound different than the oxygenated aminophenol and the benzoquinone/naphthoquinone.

The polymerizable monomer that is subjected to polymerization inhibition can include a vinyl or ethylenically unsaturated group. For example, the naphthoquinone and oxygenated oxygenated aminophenol can be added to a composition that includes one or more of the following polymerizable monomers: acrylic acid, acrylonitrile, alkylated styrene, butadiene, chloroprene, divinylbenzene, ethyl acrylate, ethyl methacrylate, isoprene, methacrylic acid, methyl methacrylate, methyl acrylate, α-methylstyrene, methacrylonitrile, styrene, styrene sulfonic acid, vinyltoluene, and vinylpyridine.

The polymerizable monomer can be present in a crude mixture of compounds, a semi-refined mixture of compounds, or a fully-refined mixture of compounds. For example, the oxygenated aminophenol and the benzoquinone/naphthoquinone can be added to a process stream that includes the polymerizable monomer. In methods, the components can be added before, during, or after, (or combinations thereof) a processing step, such as distillation, wherein compounds in the composition are separated from one another. The components can inhibit polymerization of monomer at any one or more stages in a processing system, and minimize fouling of equipment.

Alternatively, the components of the oxygenated aminophenol and the benzoquinone/naphthoquinone can be added to a process stream that includes a compound capable of the polymerizable monomer. For example, in compositions including a compound that is capable of forming a polymerizable monomer as an unwanted by-product, the presence of the oxygenated aminophenol and the benzoquinone/naphthoquinone can inhibit polymerization of the monomer if it does form as a by-product, and can therefore minimize fouling of equipment.

In some modes of practice, the oxygenated aminophenol and the benzoquinone/naphthoquinone are introduced into a monomer-containing composition to provide a desired amount of each reagent in the composition. The oxygenated aminophenol and the benzoquinone/naphthoquinone can be introduced simultaneously, such as delivered from a composition where the components are in mixture, or can be delivered individually or partially combined either sequentially, or in an overlapping manner.

If the oxygenated aminophenol and the benzoquinone/naphthoquinone are introduced into a monomer-containing composition sequentially, preferably the additions are performed to allow the oxygenated aminophenol and the benzoquinone/naphthoquinone combination to provide an unexpected (e.g., synergistic) antipolymerant effect to the composition.

The resulting introduction of the components into the monomer-containing composition can provide the oxygenated aminophenol and the benzoquinone/naphthoquinone at desired concentrations. For example, at a polymerizable monomer concentration in the range of 50 to 200 ppm, the benzoquinone/naphthoquinone is present in the composition in an amount in the range of 50 to 500 ppm, and the oxygenated aminophenol is present in the composition in an amount in the range of 50 to 500 ppm. In some modes of practice, the benzoquinone/naphthoquinone is present in the composition in an amount in the range of 75 to 400 ppm, and the oxygenated aminophenol is present in the composition in an amount in the range of 75 to 400 ppm. In some modes of practice, the benzoquinone/naphthoquinone is present in the composition in an amount in the range of 100 to 300 ppm, and the oxygenated aminophenol is present in the composition in an amount in the range of 100 to 300 ppm. In some modes of practice, the benzoquinone/naphthoquinone is present in the composition in an amount in the range of 150 to 250 ppm, and the oxygenated aminophenol is present in the composition in an amount in the range of 150 to 250 ppm.

The term “fouling” refers to the formation of polymers, prepolymers, oligomer and/or other materials which would become insoluble in and/or precipitate from a stream and deposit on equipment under the conditions of operation of the equipment. In turn, the naphthoquinone and oxygenated aminophenol and compositions of the disclosure can be referred to as “antifouling” as they inhibit or reduce such formation.

The components of the oxygenated aminophenol and the benzoquinone/naphthoquinone can be used in conjunction with compositions containing polymerizable monomers and “process equipment” such as reactors, reactor beds, pipes, valves, distillation columns, trays, condensers, heat exchangers, compressors, fans, impellers, pumps, recirculators, inter-coolers, sensors, and the like, that are associated with the process and which may be subject to fouling by monomer polymerization. This term also includes sets of these components where more than one of the components is part of a “system.”

In one preferred method of use, a composition of the disclosure with oxygenated aminophenol and the benzoquinone/naphthoquinone, and optional solvent is used with a process that involves a distillation tower that is used to separate and purify vinylic monomers. For example, in art-known processes ethylbenzene can be subjected to a catalytic dehydrogenation reaction which results in the formation of styrene. The reaction product containing styrene also contains other compounds such as aromatics like toluene and benzene, unreacted ethylbenzene, and other materials such as polymers. This mixture of compounds is generally fractionally distilled using one or more distillations towers. Typically, heat is used to help separate the components in the distillation tower. Following distillation, the fractionated components can be separated into pure product streams with higher purity.

The oxygenated aminophenol and the benzoquinone/naphthoquinone, in combination or sequentially, can be introduced into a stream leading from the reaction bed to the distillation tower, or can be directly added to the distillation tower. The inhibitor composition can be added prior to heating the monomer composition or while heating the monomer composition in the distillation tower. In some embodiments, the oxygenated aminophenol and the benzoquinone/naphthoquinone have a boiling point that is greater than a desired compound (e.g., a monomer such as styrene) subject to distillation tower and during the distillation process the desired compound is separated from the amine stabilizer by virtue of temperature difference. In embodiments, the boiling point difference between the compound of interest and the oxygenated aminophenol and the benzoquinone/naphthoquinone is about 10° C. or greater, about 15° C. or greater, about 20° C. or greater, about 25° C. or greater, about 30° C. or greater, about 35° C. or greater, about 40° C. or greater, about 45° C. or greater, or about 50° C. or greater.

Alternatively, or in addition to adding the inhibitor composition during the distillation process, the oxygenated aminophenol and the benzoquinone/naphthoquinone can be added to a distillation effluent stream, such as a purified styrene stream.

The components of the oxygenated aminophenol and the benzoquinone/naphthoquinone can be used in conjunction with a “petroleum product” which refers to any hydrocarbon product obtained from a subterranean reservoir, any product derived therefrom, or any mixture thereof. Polymerizable monomers are found in or can be chemically derived from petroleum products. Nonlimiting examples of petroleum products include but are not limited to crude oil, reduced crude oil, crude distillate, heavy oil, or bitumen, hydrotreated oil, refined oil, byproducts of petroleum product processing such as pyrolysis, hydrotreating, or phase separation, or mixtures of two or more of these. A liquid petroleum product is a petroleum product that is substantially a liquid at 20° C.

The components of the oxygenated aminophenol and the benzoquinone/naphthoquinone can be added to or can be present in a “petroleum process stream” which refers to any petroleum product disposed within petroleum process equipment in fluid contact with an interior surface thereof. The petroleum process stream can include, or can be capable of forming as a by-product, one or more polymerizable monomer. The process stream may be substantially static, such as a petroleum product disposed in a settler (separator) or storage container for a selected period of contact, such as up to two years. The process stream may be substantially dynamic, such as a liquid petroleum product disposed within a pipe during transportation of the product from a first location to a second location. In some embodiments the process stream includes one or more additional components related to petroleum processing; such components are not particularly limited.

“Petroleum process equipment” or “petroleum process apparatus” refers to a manmade item having an interior surface including a metal, further wherein one or more petroleum products are fluidly contacted with the metal for any period of time and at any temperature further as determined by context. Petroleum process equipment includes items for removing petroleum products from a subterranean reservoir, for transporting one or more petroleum products from a first location to a second location, or for separating, refining, treating, isolating, distilling, reacting, metering, heating, cooling, or containing one or more petroleum products.

In embodiments, compositions including oxygenated aminophenol and the benzoquinone/naphthoquinone are thermally stable and have antipolymerant activity in processing streams or other polymerizable monomer-containing compositions at temperatures of about 20° C. to about 400° C., for example about 100° C. to 400° C., or about 100° C. to 350° C., or about 100° C. to 300° C., or about 100° C. to 250° C., or about 100° C. to 200° C., or about 100° C. to 150° C.

In embodiments, compositions including oxygenated aminophenol and the benzoquinone/naphthoquinone can be introduced into a composition with a polymerizable monomer, such as a liquid petroleum process stream in a batch-wise, a continuous, or a semi-continuous manner. In some embodiments, the oxygenated aminophenol and the benzoquinone/naphthoquinone are introduced manually; and in other embodiments, their introduction is automated. In embodiments, the amount of the oxygenated aminophenol and the benzoquinone/naphthoquinone introduced over a selected unit of time is varied with a variable composition of the associated process stream. Such variability in dosing may be conducted manually by periodic testing of the process equipment interior surfaces, following by adjusting the amount of the composition up or down based on test results; or automatically by monitoring of one or more conditions within the interior of the petroleum process equipment and signaling the need to apply more composition to the process stream.

In some embodiments, the oxygenated aminophenol and the benzoquinone/naphthoquinone are added to a petroleum product that is a crude oil, a reduced crude oil, a heavy oil, a bitumen, a coker charge, a hydrotreater influent, a hydrotreater effluent, a flashed crude, a light cycle oil, or a diesel or naphtha refinery stream. In embodiments, the compounds are added to petroleum process equipment conventionally associated with the collecting, processing, transportation, or storage of one or more of crude oil, reduced crude oil, crude distillate, heavy oil, bitumen, coker charge, flashed crude, light cycle oil, or a diesel or naphtha refinery stream, including pipes and associated infrastructure used to fluidly connect process equipment items together to facilitate processing of a process stream disposed therein.

Equipment containing the polymerizable monomer-containing compositions that are treated with the oxygenated aminophenol and the benzoquinone/naphthoquinone can result in reduction or elimination of fouling interior surface of the equipment. In embodiments, fouling is measured as a relative increase in retention of solids within the treated composition compared to the retention of solids in untreated composition over the same time period. In embodiments, fouling is measured as a relative decrease in the weight or volume of precipitate arising from a selected period of contact of a treated process stream in an associated process equipment item, relative to the same period of contact of the process equipment with the corresponding untreated process stream. Stated differently, a reduction in fouling is a relative decrease in the measured weight or volume of solids deposited on or precipitated from process equipment contacted with the treated process stream over a selected period of time, when compared to the weight or volume of solids deposited or precipitated from an untreated process stream over the same period of time.

The oxygenated aminophenol and the benzoquinone/naphthoquinone can also inhibit unwanted polymerization and fouling of the process equipment in a primary fractionation process, light ends fractionation, non-aromatic halogenated vinyl fractionation, process-gas compression, dilution steam system, caustic tower, quench water tower, butadiene extraction, propane dehydrogenation, diesel and petrol fuel stabilization, olefin metathesis, styrene purification, hydroxyhydrocarbon purification, or delays the polymerization of resins and compositions comprising ethylenically unsaturated species.

The oxygenated aminophenol and the benzoquinone/naphthoquinone can be added at any given point in a process and at one or more locations. For example, the antifouling composition can be added directly at the inter-coolers or compressors or upstream of the inter-coolers or compressors. The oxygenated aminophenol and the benzoquinone/naphthoquinone can be added continuously or intermittently to the process equipment as required in order to inhibit or reduce fouling.

The oxygenated aminophenol and the benzoquinone/naphthoquinone can be introduced to desired systems by any suitable method. For example, it may be added in neat or a dilute solution. In some embodiments, a composition containing the oxygenated aminophenol and the benzoquinone/naphthoquinone can be applied as a solution, emulsion, or dispersion that is sprayed, dripped, poured or injected into a desired opening within a system or onto the process equipment or process condensate. In some embodiments, the composition may be added with a wash oil or an attemperation water.

After introducing the composition to process equipment, treated process equipment can be observed to have less deposition on equipment than in process equipment without addition of the composition. Reduction or inhibition in fouling can be evaluated by any known method or test. In some embodiments, the reduction or inhibition of fouling can be accessed by measuring the time it takes for a sample with and without the antifoulant composition to gel.

The following illustrative, non-limiting, examples are provided, which include details of preparation of components of the experimental compositions or show formulations for comparative purposes.

### Preparation of Oxygenated Aminophenol Compounds

Exemplary oxygenated aminophenol compounds are described in

### Synthesis of N,N-Bis-(2-Hydroxy-2-Phenyethyl)-p-Aminophenol (BHPhEPA)

The following were charged into a three-neck, 1 L round-bottomed flask with; a magnetic follower, 45.396 g (815.3 mmol) styrene oxide, 45.396 g (407.7 mmol) 4-aminophenol, and 142.804 g of toluene. Two stoppers and a condenser were fitted to the flask. The flask was placed on a heating block followed by the reflux of the reaction mixture under agitation for six hours. Following the completion of the reaction, the solvent was removed to yield a viscous gum. The product was characterized for purity and the confirmation of the structure.

Other oxygenated aminophenol compounds such as N,N-bis-(3-butoxy-2-hydroxypropyl)-p-aminophenol (BBHPhEPA), and N,N-bis[3-(2-ethylbutoxy)-2-hydroxypropyl]-p-aminophenol (BEBHPhEPA) can be prepared similar to the process of BHPEAP, see also commonly assigned WO2020/223224 (Masere and Dhawan).

### Example 1: Styrene Polymerization with 400 ppm of BHPhEPA (Comparative)

A styrene solution with a BHPhEPA concentration of 400 ppm was prepared using freshly cleaned styrene. An inhibitor removal column was used to remove the 4-tert-butylcatechol (TBC) stabilizer in the styrene to yield cleaned styrene. BHPhEPA (0.0220 g) were added into a 1-L three-neck round-bottomed flask followed by clean styrene to give a total mass of 500 g and a magnetic follower. After arming the flask with a chilled-water condenser, a thermocouple and a line through which nitrogen flowed, the flask was placed on a heating mantle. The polymerization temperature was set at 120° C. Immediately, the overhead space was swept with a constant stream of nitrogen gas. When the temperature of the solution reached 120° C., this was designated as the commencement of the polymerization reaction. A portion of the solution was retrieved, diluted with toluene and the polymer concentration determined by the precipitation method. The polymer concentration was measured and reported as percent weight. Thereafter, a portion was retrieved every 30 minutes for polymer content analysis. The total polymerization time was 2 hours. In Table 1, the polymer concentration was recorded as a function of time.

### Example 2: Styrene Polymerization with 400 ppm of p-benzoquinone (BQ) (Comparative)

Using a styrene solution treated with 400 ppm of BQ, the procedure in Example 1 was used to perform the kinetic study of the antipolymerant effect BQ. The results are shown in Table 1.

### Example 3: Styrene Polymerization with Combination of 200 ppm of BHPhEPA and 200 ppm of BQ

As illustrated in Example 1, a styrene solution of 200 ppm of BHPhEPA and 200 ppm BQ was prepared. The procedure in Example 1 was followed for the kinetics study. The polymer concentration was recorded as a function of time as shown in Table 1. The combination of BHPhEPA and BQ unexpectedly displayed surprisingly better performance than either of the two antipolymerants used on their own at concentrations of 400 ppm. The combination exhibited synergistic effect.

TABLE 1

| Time | | | 400 ppm BQ + |
|---|---|---|---|
| (minutes) | 400 ppm BHPhEPA | 400 ppm BQ | 400 BHPhEPA |
| 0 | 0.0475 | 0.048 | 0.0329 |
| 30 | 0.0418 | 0.0614 | 0.0362 |
| 60 | 0.425 | 0.0872 | 0.0374 |
| 90 | 1.28 | 0.116 | 0.0532 |
| 120 | 2.86 | 0.153 | 0.0654 |
| 150 | 3.27 | 0.184 | 0.0859 |
| 180 | 5.21 | 0.265 | 0.0943 |
| 210 | 5.55 | 0.313 | 0.119 |
| 240 | 6.39 | 0.432 | 0.137 |

Comparative performances of 400 ppm of BHPhEPA, 400 ppm of BQ, and a combination of 200 ppm of BHPhEPA and 200 ppm of BQ in styrene monomer at 120° C. under anaerobic headspace conditions are shown in Table 1.

### Example 4: Styrene Polymerization with Combination of 200 ppm of BHPhEPA and 200 ppm of t-BBQ

Using the procedure as described in Examples 1-3, reaction solutions were prepared to study the effect of combining 2-t-butyl-benzoquinone (t-BBQ). Whereas in the above-mentioned examples the headspace of the reaction solution was swept with a steady stream of nitrogen, in this study a stream of the nitrogen gas was continuously bubbled through the reaction solution. The purpose of this was to sparge oxygen from the reaction solution in addition to the headspace. According to the results shown in Table 2, the combined use of BHPhEPA and t-BBQ also unexpectedly displayed the same synergy as in Example 3.

TABLE 2

| | | Example 5 | Example 6 |
|---|---|---|---|
| Time | Example 4 | 400 ppm | 200 ppm BHPhEPA + |
| (minutes) | 400 ppm t-BBQ | BHPhEPA | 200 ppm t-BBQ |
| 0 | 0.279 | 0.32 | 0.0395 |
| 20 | 0.461 | 2.89 | 0.101 |
| 40 | 0.794 | 4.37 | 0.309 |
| 60 | 1.52 | 6.19 | 0.718 |
| 80 | 2.31 | 9.2 | 1.16 |
| 100 | 3.05 | 10.01 | 1.79 |
| 120 | 5.13 | 11.4 | 3.21 |

Comparative performances of 400 ppm of BHPhEPA, 400 ppm of t-BBQ, and a combination of 200 ppm of BHPhEPA and 200 ppm of t-BBQ in styrene monomer at 120° C. under anaerobic conditions.

1. A method to impede formation of polymer in a monomer-containing composition, the method comprising
adding to a composition comprising a polymerizable monomer or a compound capable of forming a polymerizable monomer components comprising:
a benzoquinone or naphthoquinone compound, and
an oxygenated aminophenol compound,
the oxygenated aminophenol compound being a compound of Formula I: 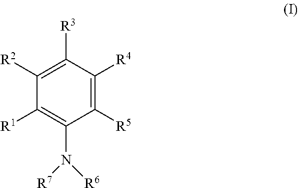
wherein at least one of —R¹, —R², —R³, —R⁴, and —R⁵ is or are —OH, and wherein any one or more of —R¹, —R², —R³, —R⁴, and —R⁵ that is not —OH is or are selected from the group consisting of hydrogen, alkyl, aryl, alkyl aryl and aryl alkyl, or any two adjacent groups of —R¹, —R², —R³, —R⁴, and —R⁵ that are not —OH form one or more ring structures;
wherein R⁶ and R⁷ are, independently, carbon-containing groups, and at least one of R⁶ and R⁷ comprises one or more oxygen atom(s) separated from the N atom by one or more carbon atoms;
wherein the benzoquinone or naphthoquinone compound, and the oxygenated aminophenol compound, impede formation of polymer in the composition.

2. The method of claim 1 wherein the oxygen atom(s) in the at least one of R⁶ and R⁷ group(s) is or are present in the form of a hydroxyl group, an ether group, or both.

3. The method of claim 1 wherein the one or more oxygen atom(s) are separated from the N atom by two or more carbon atoms.

4. The method of claim 1 wherein R⁶ or R⁷ have a number of carbon atoms of 2, or more, 3 or more, 4 or more, 5 or more, 6 or more, or in the range of 2-18, 3-17, 4-16, 5-14, or 6-12 carbon atoms, or the total number of carbon atoms in R⁶ and R⁷ is in the range of 2-30, 4-26, or 6-24 carbon atoms.

5. The method of claim 1, wherein one or both of R⁶ and R⁷ are of the formula: —R⁹OR¹⁰, wherein R⁹ comprises a hydrocarbylene group, optionally substituted, and R¹⁰ comprises a hydrocarbyl group, optionally substituted.

6. The method of claim 5 wherein (a) R⁹ is selected from the group consisting of methylene, ethylene, propylene, isopropylene, butylene, isobutylene, sec-butylene, tert-butylene, pentylene, and hexylene, (b) R¹⁰ is selected from the group consisting of linear, branched, or cyclic alkyl, aryl, alkyl-aryl, and aryl-alkyl having 1-18 carbon atoms, or both (a) and (b).

7. The method of claim 5 [or 6], wherein R⁹ is a hydroxylated hydrocarbylene group.

8. The method of claim 7 wherein R⁹ is selected from the group consisting of

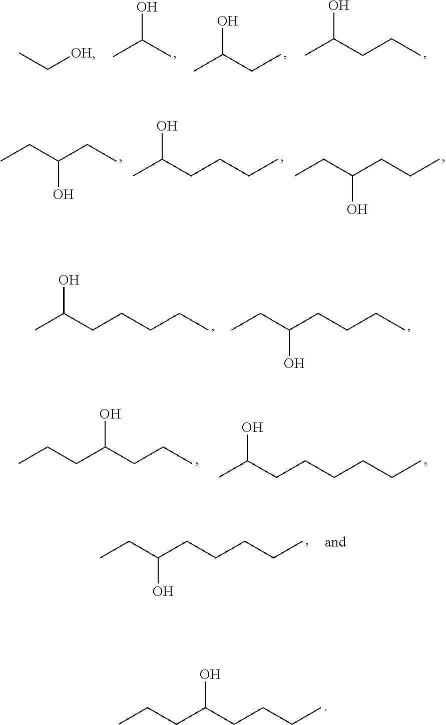

9. The method of claim 5 wherein the oxygenated aminophenol is a compound selected from the group consisting of
4-bis[(2-methoxy-1-hydroxy-ethyl)amino]phenol, 4-bis[(3-methoxy-2-hydroxy-propyl)amino]phenol, 4-bis[(4-methoxy-2-hydroxy-butyl)amino]phenol, 4-bis[(5-methoxy-2-hydroxy-pentyl)amino]phenol, 4-bis[(6-methoxy-2-hydroxy-hexyl)amino]phenol, 4-bis[(methoxyhydroxyphenyl)amino]phenol;
4-bis[(2-ethoxy-1-hydroxy-ethyl)amino]phenol, 4-bis[(3-ethoxy-2-hydroxy-propyl)amino]phenol, 4-bis[(4-ethoxy-2-hydroxy-butyl)amino]phenol, 4-bis[(5-ethoxy-2-hydroxy-pentyl)amino]phenol, 4-bis[(6-ethoxy-2-hydroxy-hexyl)amino]phenol, 4-bis[(ethoxyhydroxyphenyl)amino]phenol;
4-bis[(2-propoxy-1-hydroxy-ethyl)amino]phenol, 4-bis[(3-propoxy-2-hydroxy-propyl)amino]phenol, 4-bis[(4-propoxy-2-hydroxy-butyl)amino]phenol, 4-bis[(5-propoxy-2-hydroxy-pentyl)amino]phenol, 4-bis[(6-propoxy-2-hydroxy-hexyl)amino]phenol, 4-bis[(propoxyhydroxyphenyl)amino]phenol,
4-bis[(2-butoxy-1-hydroxy-ethyl)amino]phenol, 4-bis[(3-butoxy-2-hydroxy-propyl)amino]phenol, 4-bis[(4-butoxy-2-hydroxy-butyl)amino]phenol, 4-bis[(5-butoxy-2-hydroxy-pentyl)amino]phenol, 4-bis[(6-butoxy-2-hydroxy-hexyl)amino]phenol, and 4-bis[(butoxyhydroxyphenyl)amino]phenol.

10. (canceled)

11. The method of claim 1 wherein
(a1) the benzoquinone or naphthoquinone compound is present in the composition in an amount in the range of 50 to 5000 ppm, (b1) the oxygenated aminophenol compound is present in the composition in an amount in the range of 50 to 5000 ppm, or (c1) both (a1) and (b1); or optionally wherein
(a2) the benzoquinone or naphthoquinone compound is present in the composition in an amount in the range of 150 to 750 ppm, (b2) the oxygenated aminophenol compound is present in the composition in an amount in the range of 150 to 750 ppm, or (c2) both (a2) and (b2).

12. (canceled)

13. The method of claim 1 wherein the benzoquinone or naphthoquinone compound is added to the composition prior at the same time the oxygenated aminophenol compound is added, or the benzoquinone or naphthoquinone compound is added to the composition prior at time that is different than when the oxygenated aminophenol compound is added.

14. The method of claim 1 wherein adding is performed during purification or processing of one or more hydrocarbon components of the composition, optionally wherein processing of the one or more hydrocarbons comprises a distillation step.

15. (canceled)

16. The method of claim 1 which is performed prior to storage or transport of the composition.

17. The method of claim 1 wherein the polymerizable monomer comprises an ethylenically unsaturated group, optionally wherein the polymerizable monomer is acrylic acid, acrylonitrile, alkylated styrene, butadiene, chloroprene, divinylbenzene, ethyl acrylate, ethyl methacrylate, isoprene, methacrylic acid, methyl methacrylate, methyl acrylate, α-methylstyrene, methacrylonitrile, styrene, styrene sulfonic acid, vinyltoluene, or vinylpyridine.

18. (canceled)

19. The method of claim 1 wherein (a) the composition comprises styrene or ethylbenzene, (b) wherein the composition includes one or more non-polymerizable hydrocarbons, or both (a) and (b).

20. (canceled)

21. The method of claim 1 wherein the composition has no, less than 5 ppm, or less than 0.5 ppm, of a nitroxyl group-containing antipolymerant.

22. The method of claim 1 wherein the benzoquinone or naphthoquinone is used at a first concentration, and the oxygenated aminophenol compound is used at a second concentration, wherein the first and second concentrations together are a total concentration, and the combination of the benzoquinone or naphthoquinone and oxygenated aminophenol compound together provide a level of polymer formation that is lower than the level of polymer formation observed when either the benzoquinone or naphthoquinone and oxygenated aminophenol compound are used alone at the total concentration, or at the average of the levels of polymer formation of the benzoquinone or naphthoquinone and oxygenated aminophenol compound when used alone at the total concentration, optionally wherein the combination of the benzoquinone or naphthoquinone and oxygenated aminophenol compound impedes polymerization by an amount of 10% or greater, 20% or greater, 30% or greater, 40% or greater, 50% or greater, 60% or greater, 70% or greater, 80% or greater, or up to about 90% or greater, or a reduction in an % amount in the range of any of these numbers, as compared to the average, or as compared to either the of benzoquinone or naphthoquinone and oxygenated aminophenol compound used individually at the total concentration.

23. (canceled)

24. An additive composition for inhibiting the polymerization of monomers, the composition comprising:
a benzoquinone or naphthoquinone compound, and
an oxygenated aminophenol compound,
the oxygenated aminophenol compound being a compound of Formula I: 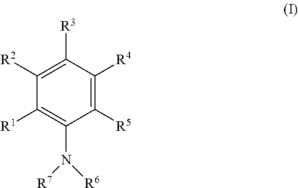
wherein at least one of —R¹, —R², —R³, —R⁴, and —R⁵ is or are —OH, and wherein any one or more of —R¹, —R², —R³, —R⁴, and —R⁵ that is not —OH is or are selected from the group consisting of hydrogen, alkyl, aryl, alkyl aryl and aryl alkyl, or any two adjacent groups of —R¹, —R², —R³, —R⁴, and —R⁵ that are not —OH form one or more ring structures;
wherein R⁶ and R⁷ are, independently, carbon-containing groups, and at least one of R⁶ and R⁷ comprises one or more oxygen atom(s) separated from the N atom by one or more carbon atoms.

25. The composition of claim 24 further comprising an organic solvent.

26. The composition of claim 25 wherein A:
the benzoquinone or naphthoquinone is in an amount in the range of 5 to 45% (wt);
the oxygenated aminophenol compound is in an amount in the range of 5 to 45% (wt); and
the organic solvent is in an amount in the range of 10 to 90% (wt); or
B:
the benzoquinone or naphthoquinone is in an amount in the range of 15 to 35% (wt);
the oxygenated aminophenol compound is in an amount in the range of 15 to 35% (wt); and
the organic solvent is in an amount in the range of 30 to 70% (wt).

27-30. (canceled)
